(19) United States
(12) Patent Application Publication
(10) Pub. No.: US 2026/0157617 A1
(43) Pub. Date: Jun. 11, 2026

(54) GUIDING LARYNGOSCOPE

(71) Applicant: Zhejiang UE Medical Corp., Taizhou (CN)

(72) Inventors: Jiazhi ZHANG, Taizhou (CN); Zhiping ZHENG, Taizhou (CN); Dongsheng WEI, Taizhou (CN); Weiping LI, Taizhou (CN); Weidong WANG, Taizhou (CN); Taohong WANG, Taizhou (CN)

(21) Appl. No.: 19/002,703

(22) Filed: Dec. 27, 2024

Related U.S. Application Data
(63) Continuation of application No. PCT/CN2024/141715, filed on Dec. 24, 2024.

(30) Foreign Application Priority Data
Dec. 9, 2024 (CN) 202411796938.9

(51) Int. Cl. A61B 1/267; A61B 1/00; A61B 1/05; A61M 16/04

(52) U.S. Cl. CPC A61B 1/267; A61B 1/00174; A61B 1/05; A61M 16/0488

(57) ABSTRACT

A guiding laryngoscope includes a holding part, a bending part and a tongue lifting part. The bending part is connected with an end of the holding part, and a guiding groove for guiding an intubation tube is provided in the bending part. One end of the tongue lifting part is connected with an end of the bending part away from the holding part and faces a side of the holding part, and the other end of the tongue lifting part is arranged to extend along a bending direction of the bending part. A first included angle between an extension line of a tube-out end of the guiding groove and a central axis of the holding part is smaller than a second included angle between an extension line of an end of the tongue lifting part and the central axis of the holding part.

### CROSS REFERENCE OF RELATED APPLICATION

This application is a continuation of and claims the benefit of priority to International Application No PCT/CN2024/141715, filed on Dec. 24, 2024, which claims the benefit of priority to Chinese Application No. 202411796938.9, filed on Dec. 9, 2024; the benefit of priority of each of which is hereby claimed herein, and which applications are hereby incorporated herein by reference in their entirety.

### BACKGROUND OF THE PRESENT INVENTION

### Field of Invention

The disclosure relates to the technical field of medical instruments, in particular to a guiding laryngoscope.

### Description of Related Arts

A laryngoscope is a kind of endoscope in the field of medical instruments, which is generally used to guide tracheal intubation. Currently, when the laryngoscope in related art is used for intubation guidance, it is often necessary for doctors to hold the laryngoscope and adjust a position of the laryngoscope several times after entering an oral cavity so as to successfully expose glottis or successfully intubate. Especially for doctors who are inexperienced in intubation, there may be more actions for adjusting, and too many adjustment actions may results in strong irritation on larynx, with difficult intubation and easy damage to throat mucosa and patient's teeth.

Therefore, it become an urgent technical problem to be solved to reduce the adjustment actions of the doctors in a process of using the laryngoscope.

### SUMMARY OF THE PRESENT INVENTION

A guiding laryngoscope is provided in the disclosure, which at least solves a technical problem of how to reduce adjustment actions of doctors in a process of using the laryngoscope.

A guiding laryngoscope is provided in an embodiment of the disclosure, which includes a holding part, a bending part and a tongue lifting part. The bending part is connected with an end of the holding part, and a guiding groove for guiding an intubation tube is provided in the bending part. One end of the tongue lifting part is connected with an end of the bending part away from the holding part and faces a side of the holding part, and the other end of the tongue lifting part is arranged to extend along a bending direction of the bending part. The guiding groove has a tube-in end and a tube-out end, and a first included angle between an extension line of the tube-out end and a central axis of the holding part is smaller than a second included angle between an extension line of an end of the tongue lifting part and the central axis of the holding part.

Optionally, difference between the second included angle and the first included angle is greater than 0° and less than or equal to 4°.

Optionally, the first included angle is 75° to 77°.

Optionally, the holding part is inclined towards a tongue lifting direction.

Optionally, the guiding laryngoscope further includes an intubation-tube positioning part provided at the tube-in end of the guiding groove, and the intubation-tube positioning part protrudes in the direction away from the holding part.

Optionally, the guiding laryngoscope further includes an intubation-tube guiding part which is arranged at the tube-out end of the guiding groove and extends along the extension line of the tube-out end.

Optionally, the guiding laryngoscope further includes a camera, which is arranged at a side of the tube-out end of the guiding groove, and a central axis of a viewing angle of the camera is inclined towards a direction of a movement axis of the intubation tube after the intubation tube extends out along the tube-out end of the guiding groove.

Optionally, a first distance between an intersection of the central axis of the viewing angle of the camera and the movement axis of the intubation tube and the holding part is adapted to a second distance between incisor and glottis.

Optionally, an included angle between the central axis of the camera and the movement axis of the intubation tube is 7° to 10°.

Optionally, the first distance is a sum of a distance between the intersection of the central axis of the viewing angle of the camera and the movement axis of the intubation tube and the end of the tongue lifting part and a distance between the end of the tongue lifting part and the holding part.

The disclosure has following beneficial effects:

The guiding laryngoscope includes the holding part, the bending part and the tongue lifting part. The bending part is connected with an end of the holding part. The bending part is internally provided with the guiding groove for accommodating movement of the intubation tube and guiding the intubation tube into the glottis. The tongue lifting part is used for exposing the glottis at epiglottis of a patient and guiding medical staff to accurately intubate the airway. One end of the tongue lifting part is connected to a side, facing the holding part, of an end of the bending part away from the holding part. The other end of the tongue lifting part is arranged to extend along the bending direction of the bending part. The holding part is used as a handle of the laryngoscope, and the handle and the tongue lifting part are connected with each other by a large-angle elliptical arc bending part. A separate cavity is defined in the bending part as an intubation-tube guiding groove, so that the intubation tube can directly enter the airway along the intubation-tube guiding groove, and operability of the guiding laryngoscope is improved. Meanwhile, the first included angle between the end of the guiding groove away from the holding part, that is, the extension line of the tube-out end of the guiding groove and the central axis of the holding part is smaller than the second included angle between an end of the tongue lifting part away from the holding part (that is, the extension line of the end of the tongue lifting part) and the central axis of the holding part. That is to say, a direction of the guiding groove at its end away from the holding part is not parallel to a direction of the tongue lifting part, and an upwarping angle of the guiding groove at its end away from the holding part is larger than an upwarping angle of the tongue lifting part at its end away from the holding part. Therefore, it is possible to correct an angle at which the intubation tube sinks due to gravity after leaving the guiding groove and meet an angle for the intubation tube to directly enter to observe the glottis through the guiding groove, and carry out airway management. Especially, when intubation is made by unskilled medical staff after aiming the guiding groove at the glottis, because the angle at which the intubation tube sinks due to gravity after leaving the tube-out end of the guiding groove is corrected, the intubation tube can directly enter the glottis without adjusting a laryngoscope angle or an intubation angle, thus reducing adjustment operations.

Further, the guiding laryngoscope further includes the camera, which is arranged at the side of the tube-out end of the guiding groove, and the central axis of the viewing angle of the camera is inclined towards the direction of the movement axis of the intubation tube after the intubation tube extends out along the tube-out end of the guiding groove. The viewing angle of the camera can be oriented to an intubation position, so as to facilitate guiding of the intubation to the glottis and ensure a success rate of intubation.

Further, the first distance between the intersection of the central axis of the viewing angle of the camera and the movement axis of the intubation tube and the holding part is adapted to the second distance between the incisor and the glottis, so that the intersection of the central axis of the viewing angle of the camera and the direction of the movement axis of intubation tube after the intubation tube extends out along the tube-out end of the guiding groove just falls at the glottis position, that is, it is ensured that a position for the intubation tube to go out along an intubation channel, the guiding groove, is just at the glottis position for viewing.

### BRIEF DESCRIPTION OF THE DRAWINGS

Accompanying drawings, which are incorporated in and constitute a part of this specification, illustrate embodiments consistent with the disclosure, and together with the specification, serve to explain principles of the disclosure.

In order to explain the embodiments of the present disclosure or the technical scheme in the prior art more clearly, the drawings required in the description of the embodiments or the prior art will be briefly introduced below; obviously, other drawings can be obtained according to these drawings by those of ordinary skill in the art without paying creative labor.

FIG. 1 is a schematic view of an exemplary structure of a guiding laryngoscope according to an embodiment of the present disclosure;

FIG. 2 is a schematic view of positions and a relationship of a first included angle and a second included angle according to an embodiment of the present disclosure;

FIG. 3 is a schematic view of an exemplary structure of another guiding laryngoscope according to an embodiment of the present disclosure; and

FIG. 4 is a schematic view of a positional relationship between a central axis of a viewing angle of a camera and a movement axis of an intubation tube according to an embodiment of the present disclosure.

### DETAILED DESCRIPTION OF THE PREFERRED EMBODIMENT

In order to make the ordinary skilled in the art better understand the schemes of the disclosure, the technical schemes in the embodiments of this disclosure will be described clearly and completely with reference to the drawings in the embodiments of this disclosure; and it is obvious that the described embodiments are only part of the embodiments of this disclosure, but not all of them. On a basis of the embodiments in this disclosure, all other embodiments obtained by the ordinary skilled in the art without any creative effort should be within the protection scope of this disclosure.

It should be noted that terms “first”, “second” and so on in the specification and claims of this disclosure and the above drawings are used to distinguish similar objects, but not necessarily to describe a specific order or sequence. It should be understood that the data thus used can be interchanged under appropriate circumstances to facilitate embodiments of the present disclosure described herein to be implemented in an order except those illustrated or described herein. In addition, terms “including” and “having” and any variations thereof are intended to cover non-exclusive inclusion, for example, a process, method, system, product or apparatus containing a series of steps or units need not be limited to those steps or units explicitly listed, but may include other steps or units not explicitly listed or inherent to these processes, methods, products or apparatus.

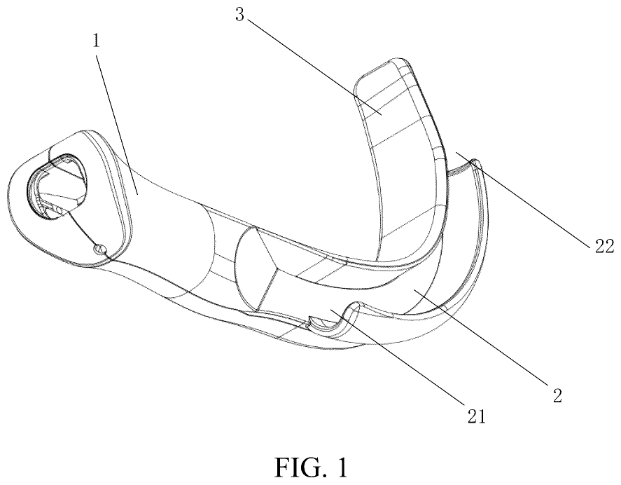

A guiding laryngoscope is provided in an embodiment of the disclosure, as shown in FIG. 1, which includes a holding part 1, a bending part and a tongue lifting part 3. The bending part is connected with an end of the holding part 1. The bending part is internally provided with the guiding groove 2 for accommodating movement of the intubation tube and guiding the intubation into the glottis. The tongue lifting part 3 is used for exposing the glottis at epiglottis of a patient and guiding medical staff to accurately intubate the airway. One end of the tongue lifting part 3 is connected to a side, facing the holding part 1, of an end of the bending part away from the holding part 1. The other end of the tongue lifting part 3 is arranged to extend along the bending direction of the bending part. The holding part 1 is used as a handle of the laryngoscope, and the handle and the tongue lifting part 3 are connected with each other by a large-angle elliptical arc bending part. A separate cavity is defined in the bending part as an intubation-tube guiding groove 2, so that the intubation tube can directly enter the airway along the intubation-tube guiding groove 2, and operability of the guiding laryngoscope is improved.

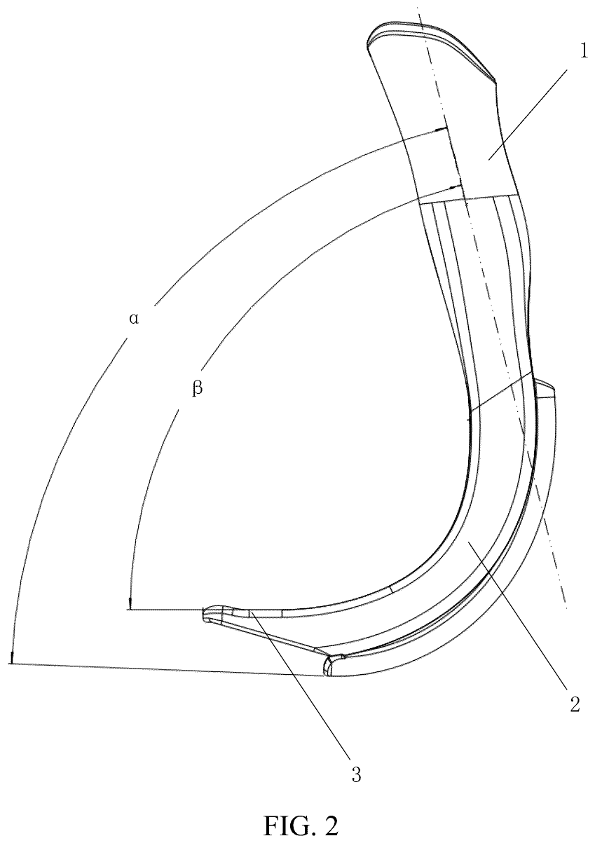

In a process of using the laryngoscope to guide the intubation tube, because the intubation tube is flexible, and there is still a distance between a tube-out end 22 of the guiding groove 2 and the glottis, the intubation tube may sink due to gravity after leaving the guiding groove 2, which may result in need to adjust a position of the laryngoscope or the intubation tube to enter the laryngoscope smoothly when intubation is made after the guiding groove 2 is aligned to the glottis. Therefore, in this embodiment, as shown in FIG. 2, an first included angle α between the end of the guiding groove 2 away from the holding part 1, that is, the extension line of the tube-out end 22 of the guiding groove 2 and the central axis of the holding part 1 is smaller than a second included angle β between an end of the tongue lifting part 3 away from the holding part 1 (that is, the extension line of the end of the tongue lifting part 3) and the central axis of the holding part 1. That is to say, a direction of the guiding groove 2 at its end away from the holding part 1 is not parallel to a direction of the tongue lifting part 3, and an upwarping angle of the guiding groove 2 at its end away from the holding part 1 is larger than an upwarping angle of the tongue lifting part 3 at its end away from the holding part 1. Therefore, it is possible to correct an angle at which the intubation tube sinks due to gravity after leaving the guiding groove 2 and meet an angle for the intubation tube to directly enter to observe the glottis through the guiding groove 2, and carry out airway management. Especially, when the intubation is made by unskilled medical staff after aiming the guiding groove 2 at the glottis, because the angle at which the intubation tube sinks due to gravity after leaving the tube-out end 22 of the guiding groove 2 is corrected, the intubation tube can directly enter the glottis without adjusting a laryngoscope angle or an intubation angle, thus reducing adjustment operations.

In an embodiment, because a distance between the tube-out end 22 of the guiding groove 2 and the glottis is small and flexibility of the intubation tube is not particularly high, difference between the second included angle β and the first included angle α is greater than 0° and less than or equal to 4°. That is, the guiding groove 2 and the tongue lifting part 3 only need to be arranged in non-parallel, and a small angle can be set to correct an angle at which the intubation tube sinks due to gravity after leaving the guiding groove 2, so as to meet an angle for the intubation tube to directly enter to observe the glottis through the guiding groove 2.

The laryngoscope in related art is often L-shaped, and an included angle between the tongue lifting part 3 and the holding part 1 is often 85° to 90°, and the holding part 1 is often vertically arranged. When the laryngoscope enters the oral cavity, an action to lift the laryngoscope is required to achieve a better glottis exposure rate for skilled doctors, while more adjustment actions are required for unskilled doctors. Therefore, in order to realize directness of observing the glottis by the laryngoscope and satisfy that the laryngoscope can quickly find the glottis position without extra actions such as lifting after directly entering the oral cavity, in an alternative embodiment, the first included angle α is set to 75° to 77°. By adapting angle setting of the first included angle α to setting of angle difference between the first included angle α and the second included angle β, after the laryngoscope enters the oral cavity, it is often possible to realize directly alignment of the intubation tube led out from the tube-out end 22 of the guiding groove 2 to the glottis without extra actions.

In an embodiment, the holding part 1 is inclined towards the tongue lifting part 3, that is, the holding part 1 and the tongue lifting part 3 are close to each other, and are connected by a large-angle elliptical arc. The holding part 1 is designed to be inclined forward, so that a viewing angle can be increased by inclining back the holding part 1 for patients who encounter difficulties in glottis exposure, which meets operability of the guiding laryngoscope and reaches a maximum exposure rate in glottis observation.

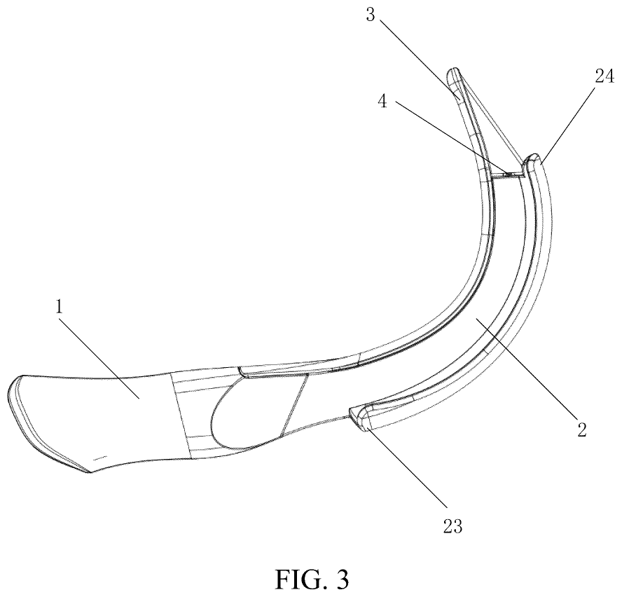

In an embodiment, as shown in FIG. 3, the guiding laryngoscope further includes an intubation-tube positioning part 23, which is arranged at a tube-in end 21 of the guiding groove 2, that is, the intubation-tube positioning part 23 is arranged at an opening of an end of the guiding groove 2 close to the holding part 1, and the intubation-tube positioning part 23 protrudes in a direction away from the holding part 1. It is ensured that the guiding groove 2 has enough space to ensure that the intubation tube can move smoothly, and that the intubation tube may not be separated from the guiding groove 2.

In an embodiment, as shown in FIG. 3, the guiding laryngoscope further includes an intubation-tube guiding part 24, and the tube-out end 22 of the guiding groove 2 extends along an extension line 221 of the guiding groove. The intubation-tube guiding part 24 is extended from the tube-out end 22 of the guiding groove 2 along the guiding groove 2, which can realize overall positioning of the intubation tube, so that when the intubation tube reaches the intubation-tube guiding part 24 along the guiding groove 2, the intubation-tube guiding part 24 can make the intubation tube enter the glottis according to an observed trajectory without being influenced by a curvature of the intubation tube itself.

In an embodiment, for convenience of observation, the guiding laryngoscope can be a visual guiding laryngoscope. The visual laryngoscope can be used to lift the exposed glottis at epiglottis of patients in clinic to guide medical staff to perform airway intubation accurately, and can also be used to provide images for oral examination and treatment. After the laryngoscope is placed into the larynx, a structure of the larynx can be directly and clearly displayed through a display screen, and the glottis can be clearly exposed, thus achieving a purpose of accurate intubation.

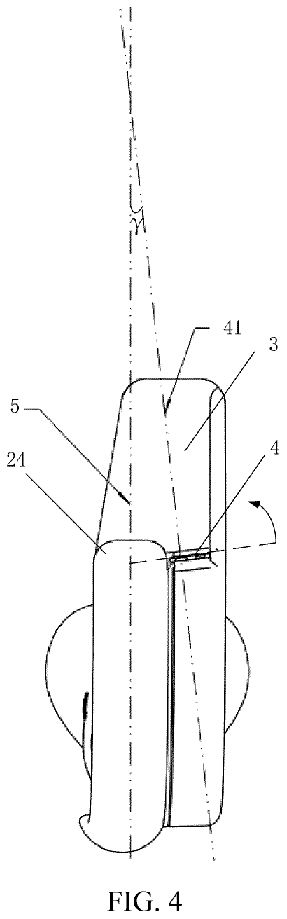

In the related art, the central axis 41 of the viewing angle of the camera 4 is often parallel to the movement axis of the intubation tube in the guiding groove 2, so that there is certain deviation between a viewing point and an intubation position. During intubation, it is necessary to move the laryngoscope to observe the intubation position, which may undoubtedly add extra operations, and especially for the unskilled doctors, more operations may be needed to repeatedly observe the intubation position before the intubation. Therefore, in this embodiment, in order to realize directness of the visual laryngoscope observing the glottis and satisfy that the glottis position can be quickly found without extra actions such as lifting after the laryngoscope directly enters the oral cavity, in this embodiment, as shown in FIGS. 3 and 4, the guiding laryngoscope further includes a camera 4, which is arranged at a side of the tube-out end 22 of the guiding groove 2, and the central axis 41 of the viewing angle of the camera 4 is inclined in the direction of the movement axis 5 of the intubation tube after the intubation extends out along the tube-out end 22 of the guiding groove 2. The viewing angle of the camera 4 can be oriented to an intubation position, so as to facilitate guiding of the intubation to the glottis and ensure a success rate of intubation. Adaptive adjustment can be made based on applicable crowd, angles and distances.

In an embodiment, as shown in FIG. 4, a third included angle γ between the central axis 41 of the viewing angle of the camera 4 and the movement axis 5 of the intubation tube is 7° to 10°, that is, the camera 4 is turned over by 7° to 10° towards the guiding groove 2 in a direction indicated by an arrow, so as to ensure that an intersection between the central axis 41 of the viewing angle of the camera 4 and the movement axis 5 of the intubation tube is located at the glottis. In this embodiment, the third included angle γ between the central axis 41 of the viewing angle of the camera 4 and the movement axis 5 of the intubation tube can be adaptively adjusted based on applicable crowd, for example, for different crowds such as children, adults and those with short mandibles, the third included angle γ can be an adaptive angle of 7°, 8°, 9° and 10°.

Further, in order to ensure that the intersection between the central axis 41 of the viewing angle of the camera 4 and the movement axis 5 of the intubation tube after the intubation extends out along the tube-out end 22 of the guiding groove 2 just falls at the glottis position, that is, to ensure that a position for the intubation tube to go out along an intubation channel, the guiding groove 2, is just at the glottis position for viewing, in this embodiment, a first distance between the intersection of the central axis 41 of the viewing angle of the camera 4 and the movement axis 5 of the intubation tube and the holding part 1 is adapted to a second distance between the incisor and the glottis.

Generally, according to a structure of a human body, the second distance between incisor and glottis of the human body is 130 mm to 150 mm, and a lower end of the holding part 1 is often abutted against the incisor. Therefore, in this embodiment, the first distance between the intersection of the central axis 41 of the viewing angle of the camera 4 and the movement axis 5 of the intubation tube and the holding part 1 is adapted to be within a second distance range, so that when the laryngoscope enters the oral cavity and the holding part 1 is abutted against the incisor, an observation viewpoint can be locked at an entrance of the glottis, and when the intubation tube reaches a focus viewpoint along the guiding groove 2, the intubation tube can just be guided to the entrance of the glottis to ensure the success rate of intubation. In this embodiment, the first distance can be adaptively adjusted based on applicable crowd, for example, the first distance can be adaptively adjusted for different crowds such as children, adults and those with short mandibles.

In an embodiment, the first distance can include two parts, a first part is a distance between the holding part 1 and an endpoint of the tongue lifting part 3 and a second part is a distance between the endpoint of the tongue lifting part 3 and the intersection of the central axis 41 of the viewing angle of the camera 4 and the movement axis 5 of the intubation tube. The first part is determined by a size of the laryngoscope, and thus after the first part is determined, the second part can be determined by adjusting the third included angle γ between the central axis of the camera 4 and the movement axis 5 of the intubation tube, so that the second part is within a second distance range or less than or equal to the second distance, and the intubation tube exactly can be guided to the entrance of the glottis when it reaches the focus viewpoint along the guiding groove 2, thus ensuring the success rate of intubation.

For example, the camera 4 of the laryngoscope is obliquely designed to form an included angle with the guiding groove 2, and the central axis 41 of the viewing angle of the camera 4 is at a middle part of the holding part 1 of the laryngoscope, and meanwhile the distance between the intersection of the movement axis 5 of the intubation tube and the central axis 41 of the viewing angle and the endpoint of the tongue lifting part 3 is controlled to be about 50 mm to 60 mm, and the holding part 1 is 70 mm away from the endpoint of the tongue lifting part 3, and according to the structure of the human body, the incisor of the human body is 130 mm to 150 mm away from the glottis, thus the focus viewpoint can be about 120 mm to 130 mm away from the handle. The included angle design of the product can smoothly lock the observation viewpoint at the entrance of the glottis. When the intubation tube goes to the observation viewpoint along the guiding groove 2, the intubation tube is exactly guided to the entrance of the glottis, ensuring the success rate of intubation.

The above are only preferred embodiments of the present disclosure. It should be pointed out that some improvements and modifications can be made by those of ordinary skilled in the art without departing from technical principle of the present disclosure, which should also be regarded to be within a protection scope of the present disclosure.

What is claimed is:

1. A guiding laryngoscope, comprising a holding part, a bending part and a tongue lifting part, wherein
the bending part is connected with an end of the holding part, and a guiding groove for guiding an intubation tube is provided in the bending part; one end of the tongue lifting part is connected with an end of the bending part away from the holding part and faces a side of the holding part, and the other end of the tongue lifting part is arranged to extend along a bending direction of the bending part; and
the guiding groove has a tube-in end and a tube-out end, and a first included angle between an extension line of the tube-out end and a central axis of the holding part is smaller than a second included angle between an extension line of an end of the tongue lifting part and the central axis of the holding part.

2. The guiding laryngoscope according to claim 1, wherein difference between the second included angle and the first included angle is greater than 0° and less than or equal to 4°.

3. The guiding laryngoscope according to claim 1, wherein the first included angle is 75° to 77°.

4. The guiding laryngoscope according to claim 1, wherein the holding part is inclined towards a tongue lifting direction.

5. The guiding laryngoscope according to claim 1, further comprising an intubation-tube positioning part provided at the tube-in end of the guiding groove, the intubation-tube positioning part protruding in the direction away from the holding part.

6. The guiding laryngoscope according to claim 1, further comprising:
an intubation-tube guiding part which is arranged at the tube-out end of the guiding groove and extends along the extension line of the tube-out end.

7. The guiding laryngoscope according to claim 1, further comprising:
a camera, which is arranged at a side of the tube-out end of the guiding groove, and a central axis of a viewing angle of the camera being inclined towards a direction of a movement axis of the intubation tube after the intubation tube extends out along the tube-out end of the guiding groove.

8. The guiding laryngoscope according to claim 7, wherein a first distance between an intersection of the central axis of the viewing angle of the camera and the movement axis of the intubation tube and the holding part is adapted to a second distance between incisor and glottis.

9. The guiding laryngoscope according to claim 7, wherein an included angle between the central axis of the camera and the movement axis of the intubation tube is 7° to 10°.

10. The guiding laryngoscope according to claim 8, wherein the first distance is a sum of a distance between the intersection of the central axis of the viewing angle of the camera and the movement axis of the intubation tube and the end of the tongue lifting part and a distance between the end of the tongue lifting part and the holding part.

11. The guiding laryngoscope according to claim 5, further comprising:
a camera, which is arranged at a side of the tube-out end of the guiding groove, and a central axis of a viewing angle of the camera being inclined towards a direction of a movement axis of the intubation tube after the intubation tube extends out along the tube-out end of the guiding groove.

12. The guiding laryngoscope according to claim 11, wherein a first distance between an intersection of the central axis of the viewing angle of the camera and the movement axis of the intubation tube and the holding part is adapted to a second distance between incisor and glottis.

13. The guiding laryngoscope according to claim 11, wherein an included angle between the central axis of the camera and the movement axis of the intubation tube is 7° to 10°.

14. The guiding laryngoscope according to claim 12, wherein the first distance is a sum of a distance between the intersection of the central axis of the viewing angle of the camera and the movement axis of the intubation tube and the end of the tongue lifting part and a distance between the end of the tongue lifting part and the holding part.

15. The guiding laryngoscope according to claim 6, further comprising:
a camera, which is arranged at a side of the tube-out end of the guiding groove, and a central axis of a viewing angle of the camera being inclined towards a direction of a movement axis of the intubation tube after the intubation tube extends out along the tube-out end of the guiding groove.

16. The guiding laryngoscope according to claim 15, wherein a first distance between an intersection of the central axis of the viewing angle of the camera and the movement axis of the intubation tube and the holding part is adapted to a second distance between incisor and glottis.

17. The guiding laryngoscope according to claim 15, wherein an included angle between the central axis of the camera and the movement axis of the intubation tube is 7° to 10°.

18. The guiding laryngoscope according to claim 16, wherein the first distance is a sum of a distance between the intersection of the central axis of the viewing angle of the camera and the movement axis of the intubation tube and the end of the tongue lifting part and a distance between the end of the tongue lifting part and the holding part.
